(19) United States
(12) Patent Application Publication
(10) Pub. No.: US 2006/0057634 A1
(43) Pub. Date: Mar. 16, 2006

(54) Assay for glycosylated proteins

(76) Inventor: Philip Rye, Oslo (NO)

Correspondence Address: BACON & THOMAS, PLLC, 625 SLATERS LANE, FOURTH FLOOR, ALEXANDRIA, VA, 22314, US

(21) Appl. No.: 10/515,172

(22) PCT Filed: May 29, 2003
(86) PCT No.: PCT/GB03/02374

§ 371 Date: Nov. 24, 2004

(30) Foreign Application Priority Data
May 29, 2002 (GB) 0212391.7

(51) Int. Cl. G01N 33/53

(52) U.S. Cl. 435/7.1

(57) ABSTRACT

The invention provides a method for assaying for a protein having at least two isoforms having different glycosylation patterns, said method comprising: contacting a sample containing said protein with a carbohydrate-binding agent and a ligand capable of binding to at least two said isoforms, and detecting conjugates of said carbohydrate-binding agent and said protein and/or of said ligand and said protein.

This invention relates to an assay for proteins having two or more isoforms differing in their pattern of glycosylation, e.g. having glycosylated and non-glycosylated isoforms or fully and partially glycosylated isoforms, and to kits for such assays.

Various proteins exist in two or more different isoforms differing in their pattern of glycosylation. Such differences, or the relative proportions of the differently glycosylated isoforms, may be indicative of a disease or disorder or of substance abuse and thus there is a need for assay systems capable of distinguishing between the differently glycosylated isoforms.

The use of antibodies to distinguish between differently glycosylated isoforms of endogenous proteins is however relatively problematic as the success rate in raising antibodies which bind specifically or preferentially to particular isoforms of endogenous glycosylated proteins is relatively low.

One example where the determination of the relative concentrations of differently glycosylated isoforms of an endogenous protein is of clinical interest is the case of the blood protein transferrin. The amino acid backbone of transferrin contains two sites (Asn 413 and Asn 611) which may bear bi- or tri-antennary oligosaccharide side chains with terminal sialic acid groups. In a healthy patient, the majority of the blood transferrin molecules carry four or five sialic acid groups; however where the patient is an alcoholic the proportion of the transferrin molecules with no sialic acid groups or with two or three sialic groups is relatively increased. (See for example Arndt in Clinical Chemistry 47: 13-27 (2001)). Abnormal relative abundances of the transferrin isoforms also occur in patients with carbohydrate-deficient glycoprotein syndromes (CDGS) or congenital disorders of glycosylation (CDG), e.g. as discussed by Keir et al. in Ann. Clin. Biochem. 36: 20-36 (1999).

Various assays for such “carbohydrate-deficient transferrin” (CDT) or “carbohydrate-free transferrin” (CFT) have been proposed; however those suitable for automation generally rely on the use of an ion exchange resin to separate out the transferrin molecules with three or less sialic acid groups from those with four or five sialic acid groups on the basis of the different pHs at which the different isoforms are released from or taken up by the resin. Examples of such assays are described in U.S. Pat. No. 4,626,355 (Pharmacia), WO 96/26444 (Axis) and WO 01/42795 (Axis).

Any protein with post-translational glycosylation can occur in different glycosylation isoforms. Thus, besides transferrin other clinically relevant proteins exist in differently glycosylated isoforms, including glycosylated markers for cancers and other diseases, e.g. alkaline phosphatase (AP) (see Magnusson et al. Clinical Chemistry 44: 1621-1628 (1998)), alpha-fetoprotein (AFP), human chorionic gonadotropin (HCG), and possibly also prion protein (CD230).

Mammalian alkaline phosphatases comprise a ubiquitous family of enzymes. AP is a glycoprotein enzyme, residing in the outer leaflet of the cytoplasmic membrane where a glycosyl phosphatidylinositol moiety serves as a membrane anchor. The (native) molecular mass of liver AP, bone AP, and kidney AP has been determined as 152, 166 and 168 kDa respectively. Apart from its role in normal bone mineralization, other functions of L/B/K AP in physiological and neoplastic conditions remain unknown. Alkaline phosphatase is present in human serum in several isoforms. Identification of the different isoforms in serum is complicated by the variety of post-translational modifications. The two major circulating AP isoenzymes, bone and liver, are difficult to distinguish because they are the products of a single gene and differ only by glycosylation. Total serum AP is frequently requested in routine clinical analyses, to determine skeletal and hepatobiliary status. It has been suggested that the various isoforms contributing to the total AP activity provide useful clinical information. Indeed quantitative measurement of bone AP (BAP) activity in serum can provide an index for the rate of bone formation.

Alpha-fetoprotein (AFP) is a major protein of mammalian fetal development and is synthesized mainly by fetal liver and yolk sac. Since hepatoma and yolk sac tumors often produce this protein, it has routinely been used as a tumor marker for diagnosis. In particular AFP is widely used as a serological marker in the diagnosis of hepatocellular carcinoma (HCC) and non-seminomatous germ cell tumours (NSGCT). AFP is also elevated in normal pregnancy, benign liver disease as well as cancer. AFP appears in several disease-associated isoforms that differ in carbohydrate structures. Existing assays cannot easily differentiate between these isoforms.

Other glycoproteins of interest for the present invention include: alpha-1-acid glycoprotein, alpha-1-antitrypsin, haptoglobin, thyroglobulin, prostate specific antigen, HEMPAS erythrocyte band 3 (this is associated with congenital dyserythropoietic anemia type II), PC-1 plasma-cell membrane glycoprotein, CD41 glycoprotein IIb, CD42b glycocalicin, CD43 leukocyte sialoglycoprotein, CD63 lysosomal-membrane-associated glycoprotein 3, CD66a biliary glycoprotein, CD66f pregnancy specific b1 glycoprotein, CD164 multi-glycosylated core protein 24, and the CD235 glycophorin family.

We have now found that the problem of using antibodies or other ligands to discriminate between differently glycosylated protein isoforms in assays may be addressed by the additional use in such assays of carbohydrate-binding agents which serve to mask antibody/ligand binding sites common to differently glycosylated isoforms of the protein, i.e. where a carbohydrate side chain is present in the isoform the binding of the agent will hinder subsequent binding of an antibody (or other protein binding moiety) which is capable of binding to the protein, glycosylated or not, in the absence of the carbohydrate-binding agent.

Thus viewed from one aspect the invention provides a method for assaying for a protein having at least two isoforms having different glycosylation patterns, said method comprising contacting a sample containing said protein with a carbohydrate-binding agent and a ligand capable of binding to at least two said isoforms, and directly or indirectly detecting conjugates of said carbohydrate-binding agent and said protein and/or of said ligand and said protein.

Viewed from a further aspect the invention provides a kit for an assay method according to the invention, said kit comprising a carbohydrate-binding agent and a protein binding ligand.

The kit of the invention preferably also contains a substrate having bound thereon a secondary ligand capable of binding at least two and preferably all of the isoforms of the glycoprotein. This secondary ligand is preferably one which binds the glycoprotein at a site remote from the glycosylation sites. In an especially preferred embodiment, this secondary ligand is immobilized on a porous membrane.

The kit also preferably contains instructions for the performance of the assay method and may optionally contain further, optionally labelled, secondary ligands capable of binding to the glycoprotein:primary ligand conjugate and/or the glycoprotein:carbohydrate-binding agent conjugate.

In the assay method of the invention the protein may be contacted with the carbohydrate-binding agent and the ligand simultaneously or sequentially. Sequential contact, with the contact with the carbohydrate-binding agent occurring first, is preferred. Where sequential contact is used, the protein is not separated (ie deconjugated) from the first binding reagent before the second one is applied, although any unbound excess of the first binding reagent may of course be removed if desired.

The detection of the conjugates formed by the protein may, as stated above, be direct or indirect. Thus a property (e.g. radiation absorption, emission, or scattering) of a conjugate or of the carbohydrate-binding agent or ligand may be detected, or a further binding reagent with a detectable property or the ability to provoke a detectable property or event may be used. This further binding reagent would be one which binds to such protein conjugates or competes with such protein conjugates in binding to a further substrate. Such direct and indirect detection of analytes by the use of optionally labelled binding reagents is conventional in the field of diagnostic assays.

The manner in which detection of the conjugates is made will of course be dependent on the nature of the binding reagents, i.e. whether they are labelled with a reporter moiety such as a radiolabel, a chromophore or a fluorophore, whether they are enzymatically active (i.e. capable of catalysing a reaction the progress whereof is detectable, e.g. by generation of light or a detectable species), whether they form aggregates which can be detected by light scattering, etc. Such detection systems are conventional in the field of diagnostic assays.

The carbohydrate-binding agent used in the assay method of the invention may be any species capable of binding to the carbohydrate side chains of glycoproteins and thus masking epitopes on the protein backbone. Thus the carbohydrate-binding agent may for example be a small molecule with a highly charged functional group or more preferably it may be a macromolecule. By “macromolecule” in this context is meant a compound having a molecular weight in excess of 500 D, preferably in excess of 1000 D, e.g. 500 to 100000 D, preferably 1000 to 20000 D.

The carbohydrate-binding agent is preferably a compound soluble in water or a water-miscible organic solvent, or a mixture thereof. Particularly preferably the carbohydrate-binding agent used in the assay of the invention is a peptide (e.g. a protein or other polypeptide or an oligopeptide); however other macromolecules capable of binding to carbohydrate groups may be used. Such compounds may be found using routine chemical techniques, such as library panning (e.g. of oligopeptide display libraries such as phage display libraries or of chemical libraries, for example produced using combinatorial techniques). However many carbohydrate-binding macromolecules are known from the literature, one particular example being the group of proteins known as lectins.

Lectins are proteins or glycoproteins of non-immunoglobulin nature that incorporate one or more (frequently two) binding sites that are highly specific for carbohydrate moieties.

Lectins occur in the tissues of most living organisms and were first discovered in plant extracts by their ability to agglutinate cell types based on their blood group activity. Although the term “lectin” was first used to define these agglutination activities, the term is more generally used to cover sugar-binding proteins from many sources regardless of their ability to agglutinate cells.

Most lectins studied to date are multimeric, consisting of non-covalently associated subunits. A lectin may contain two or more of the same subunit, such as Concanavalin A (or Con A, from Canavalia ensiformis), or different subunits, such as Phaseolus vulgaris agglutinin. It is this multimeric structure which gives lectins their ability to agglutinate cells or form precipitates with glycoconjugates. Although most lectins can agglutinate some cell types, cellular agglutination is not a prerequisite. Some lectins can bind to cells and not cause agglutination, such as succinylated Con A, and some lectins may not bind to cells at all. This inability to bind cells may be a consequence of the structure of the lectin or the absence of a suitable receptor oligosaccharide on the cell surface. Since agglutination of cells is the assay most generally employed to detect lectins, many non-agglutinating lectins may exist in nature which have not yet been detected.

Because of the specificity that each lectin has toward a particular carbohydrate structure, even oligosaccharides with identical sugar compositions can be distinguished or separated. Some lectins will bind only to structures with mannose or glucose residues, while others may recognize only galactose residues. Some lectins require that a particular sugar be in a terminal non-reducing position in the oligosaccharide, while others can bind to sugars within the oligosaccharide chain. Some lectins do not discriminate between a and b anomers, while others require not only the correct anomeric structure but a specific sequence of sugars for binding.

Thus where a lectin is to be used in the assay method of the invention it should be selected from the group of lectins capable of binding to a carbohydrate side chain of the protein being assayed for. Where the protein has more than one type of carbohydrate side chain, two or more different lectins having the ability to bind to different carbohydrate side chains in the protein may be used. Suitable lectins may thus be chosen from these known binding abilities or by screening for binding ability for the different isoforms of the protein. Where the desired format of the assay method involves determination of total carbohydrate-binding agent:protein conjugate content, the carbohydrate-binding agent (e.g. lectin) may be labelled with a reporter moiety, e.g. a radiolabel, chromophore or fluorophore.

Examples of currently available lectins include: AAA—Allium oscalonicum agglutinin (shallot); AAA—Aloe arborescens agglutinin (Kidachi aloe, narrow leaved sword aloe); AAA—Artocarpus altilis agglutinin; AAA, AAnA—Anguilla anguilla agglutinin (freshwater eel); AAurA—Aleuria aurantia agglutinin (orange peel fungus); AAusA—Androctonus australis agglutinin (Saharan scorpion); ABA, AbiA, ABL—Agaricus bisporus agglutinin (mushroom); ABrA—Amphicarpaea bracteata agglutinin (hog peanut); ACA—Allium cepa agglutinin (onion); ACA—Alocasia indica lectin; ACA—Amaranthus caudatus agglutinin (amaranth, tassel flower, inca wheat); ACL—Amaranthus cruentus lectin (red amaranth, purple amaranth); ACmA—Arisaema curvatum lectin; AFA—Afimbrial adhesin (bacteria); AGL—Aplysia gonad lectin; AIA—Artocarpus integrifolia agglutinin (Artocarpus heterophyllus, Indian jaca tree, jackfruit); ALA—Artocarpus lakoocha agglutinin (lakoocha, small jack, monkey fruit); AlloA—Allomyrina dichotoma agglutinin (Japanese beetle); AMA—Allium moly agglutinin (dwarf flowering onions); AMA—Arum maculatum agglutinin (lords and ladies); AQN—spermadhesin; APA—Aaptos papillata agglutinin; APA—Abrus precatorius agglutinin (jequirity bean, coral bead plant, lucky bean, crab's eyes); APA/APL—Aegopodium podagraria agglutinin/lectin (ground elder, achweed); APA—Allium porrum agglutinin (leek); APL—Aquathanatephorus pendulus lectin; ARA—Agropyrum repens agglutinin (couch grass); AREL—Agropyrum repens embryo lectin (couch grass); ARL—Athelia rolfsii lectin; ARLL—Agropyrum repens leaf lectin (couch grass); ASA/ASL—Allium sativum agglutinin/lectin (garlic, garden rocambole); ASL—Amaranthus spinosus agglutinin (thorny pigweed, spiny amaranth); AUA—Allium ursinum agglutinin (ramson, bears garlic); AVA—Allium vineale agglutinin (crow garlic); AWN—spermadhesin; BanLec—Banana lectin (Musa paradisiac); BCL—Botrytis cinerea lectin; BDA—Bryonia dioica agglutinin (white bryony); BfL—Butea frondosa lectin (Butea monosperma, bastard teak, flame of the forrest); BGA—Biomphalaria glabrata agglutinin; Blec—bud lectin (Pisum sativum); BLA—Birgus latro agglutinin (coconut crab); BMA—Bowringia milbraedii agglutinin; BPA—Bauhinia purpurea agglutinin (camels foot tree, purple mountain ebony); BSA/BSL/BSI/BSII—Bandeiraea simplicifolia agglutinin/lectin/isolectin (Griffonia simplicifolia); BsyL—Brachypodium sylvaticum lectin (false brome grass); CA—Cymbidium agglutinin; CAA—Caragana arborescens agglutinin (Siberian pea tree); CAA/CPA—Cicer arietinum agglutinin (chick pea, ceri bean); CAA/CAL—Colchicum autumnale agglutinin/lectin (meadow saffron); CBL—Cyphomandra betacea lectin (tamarillo fruit, tree tomato); CBP-35-Lactosamine-binding protein (mouse fibroblasts); CBP-67—Carbohydrate-binding protein (rat liver nuclei); CBP-70—Carbohydrate-binding protein (HL60 cell nuclei); CCL—Ceratobasidium cornigerum lectin; CD-MPR—Cation dependent mannose-phosphate receptor; CEA—Colocasia esculenta lectin (taro); CGA—Canavalia gladiata lectin (Japanese Jack bean); CGA—Canna generalis lectin; CHA—Cepaeae hortensis agglutinin (snail); CHA—Cymbidium hybrid lectin; CIA—Coccinia grandis lectin (C. indica, C. cordifolia, Ivy gourd, scarlet gourd); CI-MPR—Cation independent mannose-phosphate receptor; CLA—Cladrastis lutea lectin (Yellow wood); CLA—Clivia miniata agglutinin (Clivia); CLC—Charcot-Leyden crystal protein; CLL—Chicken lactose-binding lectin; CMA—Chelidonium majus agglutinin (celandine, greater celandine); CMA—Clivia miniata lectin; CMA—Cucurbita maxima agglutinin (marrow, winter squash); CMA—Cytisus multiflorus agglutinin; Con A—Concanavalin A (Canavalia ensiformis, jack bean); CPA—Cucurbita pepo agglutinin (pumpkin, summer squash, gourd); CRA—Carcinoscorpin (Carcinoscorpius rotunda); CRCA—Carcinoscorpius rotunda cauda (Indian horseshoe crab); CS, CSA, CSA-II, CSL—Cytisus scoparius agglutinin (Sarothamnus scoparius, Scotch broom); CSA, CSA-I, CSL—Cytisus sessilifolius agglutinin (Portugal broom); CSL—Cerebellar soluble lectin, cell-sealing lectin; CTA—Clerodendron trichotomum lectin; CTL—Croton tiglium lectin (croton); DBA—Dolichos biflorus agglutinin (horse gram); DGA—Dioclea grandiflora lectin; DIA—Datura innoxia agglutinin; DLA, LPA—Dolichos lablab agglutinin (Lablab niger, Lablab purpureus, Hyacinth bean, lablab bean, black seeded kidney bean); DSA—Datura stramonium agglutinin (Jimson weed, thornapple); EBL—Elderberry lectin (Sambucus nigra agglutinin elderberry, eldertree, elder); ECA, ECorA—Erythrina corallodendron agglutinin (West Indian coral tree); ECA, ECL—Brythrina cristagalli agglutinin (cocks comb coral tree); EEA—Euonymus europaeus agglutinin (prickwood, spindle tree); EHA—Epipactis helleborine agglutinin (broad leaved helleborine); EHA, EHL—Eranthis hyemalis lectin (winter aconite); EHA—Euphorbia heterophylla agglutinin (Mexican fire plant, painted spurge); GBL—Glucan-binding lectin (Streptococcus sp.); GCA—Geodia cydonium agglutinin; GMP-140—Platelet granule membrane protein-140, p-selectin; GNA—Galanthus nivalis agglutinin (snowdrop); GNL—Peanut nodule lectin (Arachis hypogaea); GPA—Gonatanthus pumilus agglutinin; GS, GSA—Griffonia simplicifolia agglutinin (now Bandeirea simplicifolia agglutinin); GSL—Gerardia savaglia lectin (false foxglove); HAA—Helix aspersa agglutinin (garden snail); HAA—Homarus americanas agglutinin (lobster); HCA—Hura crepitans agglutinin (sand-box tree); HHA—Hippeastrum hybrid agglutinin (amaryllis); HL-3, HL-13-Human lectins; L-29, HL-29-Lactosamine-binding protein (human lung); HPA—Helix pomatia agglutinin (Roman snail, edible snail); HTA—Helianthus tuberosus lectin (Jerusalem artichoke); HVA—Hordeum vulgare lectin (barley); IAA—Iberis amara agglutinin (candy tuft); IRA—Iris hybrid lectin (Dutch iris); JFL—Jackfruit lectin (Antocarpus heterophyllus, bread fruit tree); L-I, L-II—Leaf lectins from Winged bean (Psophocarpus tetragonolobus, goa bean, winged pea); L-34-beta-galactoside-specific lectin (mouse fibrosarcoma); LAA, LAL, LALA—Laburnum alpinum agglutinin (Scotch laburnum); LAA—Leptospermum archinoides agglutinin (Australian tea tree); LAA—Leucojum aestivum agglutinin (snowflake, summer snowflake); LAA—Luffa acutangula agglutinin (ridge gourd); LAL—Laelia autumnalis lectin; LAM-14—Mouse lymphocyte homing receptor; LANA—Laburnum angyriodes agglutinin (laburnum); LBA, LBL, PLA—Lima bean agglutinin (Phaseolus limensis, Phaseolus lunatus); LBP—Laminin-binding protein (mouse macrophages); LCA, LcH—Lens culinaris agglutinin (lentil); LCL—Litchi chinensis lectin; LcLI, II—Lathyrus cicera isolectins (dwarf chicling vetch, vetch); LEA, LEL, TL—Lycopersicon esculentum agglutinin (tomato); LEC-CAM—Selectins, group of C-type lectins; LEL—Loranthus europaeus lectin (loranthus, misteltoe); LFA—Limax flavus agglutinin; LL1 Lymphocyte lectin 1 (mammals); LNA—Lablab niger agglutinin; LOA—Lathyrus odoratus lectin (sweet pea); LOA1, 2—Listera ovata (twayblade); LoLI, II—Lathyrus ochrus isolectins (yellow flowered pea); LPA, DLA—Lablab purpureus agglutinin (Lablab niger, Dolichos lablab, Hyacinth bean, lablab bean, black seeded kidney bean); LPA—Lathyrus pratensis agglutinin (bastard vetchling, meadow lathyrus); LPA—Limulin (Limulus polyphemus, horseshoe crab); LSA—Lathyrus sativum agglutinin (chicling vetch); LTA—Lotus tetragonolobus agglutinin (lotus, birds foot trefoil, also Tetragonolobus purpurea, winged pea, asparagus pea); LtubL—Lathyrus tuberosus tuber lectin (tuberous lathyrus); LtuLI, II— Lathyrus tuberosus seed isolectins (tuberous lathyrus); LVA—Leucojum vernum agglutinin (snowflake, spring snowflake); Mac-2—Macrophage surface antigen, major non-integrin laminin-binding protein (human, mouse); MAA, MAH, MAHS, MAL—Maackia amurensis agglutinin/lectin; MBA—Machaerium biovulatum agglutinin; MBA—Mung bean agglutinin (Vigna radiata, Phaseolus aureus); MBP—Maltose-binding protein (animals); MBP—Mannan-binding protein (animals); MBP-A—Mannose-binding protein A (rat); MCA—Momordica charantia agglutinin (bitter pear melon, bitter gourd); ME-C2, ME-D2, ME-E2, ME-F2—Machaerocereus eruca isolectins; MEA—Machaerocereus eruca lectin; MEL-14-Mouse lymphocyte homing receptor; MGA—Mycoplasma gallisepticum agglutinin; mGBP—Mouse galactose binding protein; MIA—Mangifera indica agglutinin (mango tree); ML, VAA—Mistletoe lectin (Viscum album); MLA—Macharium lunatus agglutinin; MMA, MML—Marah macrocarpus lectin (wild cucumber); MMR—Macrophage mannose receptor (animals); MNL—Peanut nodule and cotyledon lectin (Arachis hypogea); MPA—Maclura pomifera agglutinin (maclura, osage orange, hedge apple tree); MPR—Mannose-phosphate receptor (animals); MT LEC1—Medicago truncatala lectin; NFA—Nonfimbrial adhesin (bacteria); NFL—Neoregelia flandria lectin; NLA—Narcissus lobularis agglutinin; NPA/NPL—Narcissus pseudonarcissus agglutinin/lectin (daffodil); OSA, RL—Oryza sativa agglutinin (rice); PA-I, PA-II—Pseudomonas aeruginosa lectins; PAA, Pa-1,2,3,4,5-Phytolacca americana isolectins (pokeweed, pigeon berry); PAA—Percea americana agglutinin (avocado); PALL—Phragmites australis lectin (common reed); PADGEM—Platelet granule membrane protein-140, p-selectin; PCA—Phaseolus coccineus agglutinin (scarlet runner bean); PHA—Phytohemagglutinin (Phaseolus vulgaris, red kidney bean); PHA-E—Erythroagglutinating isolectin of PHA; PHA-L—Leucoagglutinating isolectin of PHA; PL—Pseudamonas lectin; PLA, LBA, LBL—Phaseolus limensis agglutinin (P. lunatus, lima bean); PMA—Polygonatum multiflorum lectin (common Solomon's seal); PNA—Arachis hypogaea agglutinin (peanut); Po66-CBP—Beta-galactoside-binding lectin in lung carcinoma; PPA—Ptilota plumosa agglutinin (red marine algae); PRA—Peanut root lectin (Arachis hypogea); PRA—Pterocarpus rhorii agglutinin; PSA, PsA—Pisum sativum agglutinin (garden pea, common pea); PsNlec-1—Pisum sativum nodule lectin 1 (garden pea, common pea); PTA, PTL, WBA—Psophocarpus tetragonolobus agglutinin (goa bean, winged pea); PWM—Poke weed mitogen (Phytolacca americana); R1—Receptro 1, recognin 1; RaRF—Ra reactive factors (mammalian serum); RCA, RCAl20, RCL I, RCL II—Ricinus communis agglutinin (castor oil bean); RCA6O, RCL III, RCL IV—ricin, ricin D, ricin E (Ricinus communis, castor bean, ricin); RCL—Rhizoctonia crocorum lectin; RL, OSL—Rice lectin (Oryza sativa); RL-29-Lactosamine-binding protein (rat lung); RPA, RPsA—Robinia pseudoaccacia seed agglutinin (black locust, false acacia); RpbA—Robinia pseudoaccacia bark agglutinin (black locust, false acacia); RSA—Rhizoctonia solani lectin; SAP—Serum amyloid protein (mammals); SBA—Soybean agglutinin (Glycine max, soya bean); SCA—Sambucus canadensis lectin (Canadian elderberry); SCA—Secale cereale lectin (rye); SEA—Sambucus ebulus lectin (dward elder); SER—Sheep erythrocyte receptor (mouse macrophages); SGA—Sauromatum guttatum agglutinin; SGL —Sarcocystis gigantea lectin; SHA—Salvia horminum lectin (salvia); SJA, SJAbg/SJAbm—Sophora japonica agglutinin (Japanese/Chinese pagoda tree); SL—Onobrychis viciifolia lectin (sanfoin); SML—Sarcocystis muris lectin; SML—Sclerotinia minor lectin; SNA—Sambucus nigra agglutinin (elderberry, eldertree, elder); SP-A—Pulmonary surfactant protein-A (mammals); SRA—Sambucus racemosa lectin (red-berried elder); STA—Solanum tuberosum agglutinin (potato); SSA —Salvia sclarea agglutinin (clary, fetid clary sage); SSA—Sambucus sieboldiana lectin (Japanese elderberry); SSA—Soybean seedling agglutinin; SSA—Stenostylis stenocarpa agglutinin; SML—Sclerotinia sclerotiorum lectin; SVAK—Snake venom agglutinin (Naja naja kaouthia); SVAM—Snake venom agglutinin (Naja mossambica mossambica); SWA—Sarothamnus welwitschii lectin (broom); TAA—Thorn apple agglutinin (Datura stramonium, Jimson weed); TCA—Tetracarpidium conophorum lectin (Nigerian walnut); TKA—Trichosantes kirilowii agglutinin (serpent cucumber); TL, LEA, LEL—Tomato lectin (Lycopersicon esculentum); TL, TxLC, TXLM—Tulipa lectins (tulip); TPA—Tetragonolobus purpurea agglutinin (winged pea, asparagus pea, also Lotus Tetragonolobus, lotus, birds foot treefoil); TxLC-I, TL—Tulipa lectin (tulip); TxLM-I, TxLM-II—Tulipa lectins (tulip); UDA—Urtica dioica agglutinin (stinging nettle, nettle); URA—Ulex europaeus agglutinin (furze, gorse); VAA, ML—Viscum album agglutinin (mistletoe); VCA—Vicia cracca lectin (common vetch); VEA—Vicia ervilia lectin (bitter vetch); VFA—Favin, Vicia faba agglutinin (broad bean, garden bean); VGA—Vicia graminea agglutinin; VRA—Vigna racemosa agglutinin; VSA—Vicia sativa agglutinin (tare, vetch); VVA, VVL—Vicia villosa agglutinin (hairy vetch); WBA, PTA, PTL—Winged bean agglutinin (Psophocarpus tetragonolobus, goa bean, winged pea); WBTL—Winged bean tuber lectin (Psophocarpus tetragonolobus, goa bean, winged pea); WGS-1—Winged bean green shell lectin (Psophocarpus tetragonolobus, goa bean, winged pea); WFA, WFH—Wisteria floribunda agglutinin (Japanese wisteria); WGA—Wheat germ agglutinin (Triticum vulgare); XL35—Xenopus laevis oocyte lectin; and ZMA—Zea mays lectin (corn, maize).

The primary ligand used in the assay of the invention may be any compound capable of binding to the protein when unconjugated by the carbohydrate-binding agent but with reduced capability or no capability to bind to the protein:carbohydrate-binding agent conjugate for at least one isoform. Typically the primary ligand will be an antibody or antibody fragment, an oligopeptide, an oligonucleotide or a small organic molecule. Antibodies and antibody fragments are preferred, especially monoclonal antibodies. The primary ligand may if desired be labelled, e.g. with a radiolabel, chromophore or fluorophore. The primary ligand may be selected by selecting ligands capable of binding to the carbohydrate carrying isoform(s) of the protein and to the carbohydrate deficient isoform(s) of the protein, e.g. by raising antibodies to such proteins or fragments thereof, or to immunogenic conjugates of such proteins or fragments, or by library screening.

In one particular embodiment, antibodies may be raised against immunogenic conjugates of oligopeptides having sequences corresponding to (or similar to) part of the amino acid sequence of the protein, especially a part overlapping with or adjacent (e.g. within 10 amino acid residues of) a glycosylation site on the protein. Such oligopeptides may themselves be glycosylated and will typically be 8 to 50 amino acid residues in length, e.g. 10 to 30 residues.

Selected candidates may then be screened against the protein:carbohydrate-binding agent conjugates to identify ligands suitable for use as the primary ligand in the assay method of the invention.

Depending on the format of the assay method of the invention, performance of the assay method may involve the additional use of two or more secondary ligands. Thus a secondary ligand capable of binding all isoforms of the protein may be used to concentrate or separate the protein from the rest of the sample. Typically this may involve contacting the sample with such a ligand bound to a substrate and preferably separating the substrate from the remaining part of the sample, e.g. by washing the substrate. The substrate may take any convenient form, e.g. a plate, rod, bead, fibre or a surface coating on a tube or container. Particularly preferably the substrate is a magnetically displaceable polymeric bead, e.g. a bead containing superparamagnetic crystals. Such magnetic beads are available commercially, e.g. from Dynal Biotech, Oslo, Norway.

Other secondary ligands may be used to generate a detectable species or event so as to allow the content or relative content of the carbohydrate deficient isoforms of the protein to be determined. Such secondary ligands will typically be ligands which bind to the protein:carbohydrate-binding agent or protein:primary ligand conjugates, eg to binding sites on the protein, carbohydrate-binding agent or primary ligand exposed in the conjugates.

Labelling of the primary or secondary ligand or the carbohydrate-binding agent may be effected using conventional synthetic chemical techniques, eg by reacting the ligand or carbohydrate-binding agent, optionally after activation thereof, with a bifunctional linking agent and the label species or with an activated label species or the conjugate of the label species and a bifunctional linking agent.

In one embodiment of the invention, detection may be effected using surface plasmon resonance (SPR), a non-invasive optical technique in which the SPR response reflects the change in mass concentration at the detector surface as molecules bind or dissociate. Thus a surface bound glycoprotein, exposed first to a lectin and then to a primary ligand will generate an SPR response in the case where the lectin has prevented the primary ligand from binding which is different to the response (where the glycoprotein is carbohydrate deficient and lectin binding has not occurred) where the primary ligand is able to bind. Surface binding of the glycoprotein in this case can be achieved by using substrate bound ligands (eg antibodies) which bind to a region of the glycoprotein remote from the glycosylation sites.

SPR may be carried out using the proprietary system known as Biacore analysis (available from Biacore AB, Uppsala, Sweden).

The method of the invention is particularly suited for use in assaying multiple samples, eg using a multiwell microtitre plate format (typically an n×m well plate where n and m are positive integers having values up to 20, especially a 96-well microtitre plate).

In the assay method of invention, carbohydrate deficiency can be determined quantitatively, semi-quantitatively or qualitatively (eg as being below or above a predetermined value indicative of a boundary between normality and abnormality or between mild and severe disease states). Generally however it will be preferred to represent carbohydrate deficiency as the percent (eg mole percent) of the isoforms present that are carbohydrate deficient. To this end the assay method of the invention preferably involves a determination of total content of the glycoprotein, eg by a parallel performance of the assay without the use of the carbohydrate-binding agent.

The samples used in the assay method of the invention will typically be samples of or derived from a body tissue, organ or fluid (eg urine, saliva, mucous, blood, etc). Preferably the sample is blood or derived from blood, eg serum. The species of the subject from which the sample is taken is preferably a mammalian, reptilian, avian or fish or shellfish species, more preferably mammalian (especially human).

Where the glycoprotein is cell bound or cell-encapsulated the sample may be treated in conventional fashion to release the glycoprotein. Similarly the glycoprotein may if desired be metallated (eg by addition of iron ions where the protein is an iron-binding protein), demetallated or denatured. The precise nature in which the sample is pretreated will thus depend on the particular glycoprotein being assayed for.

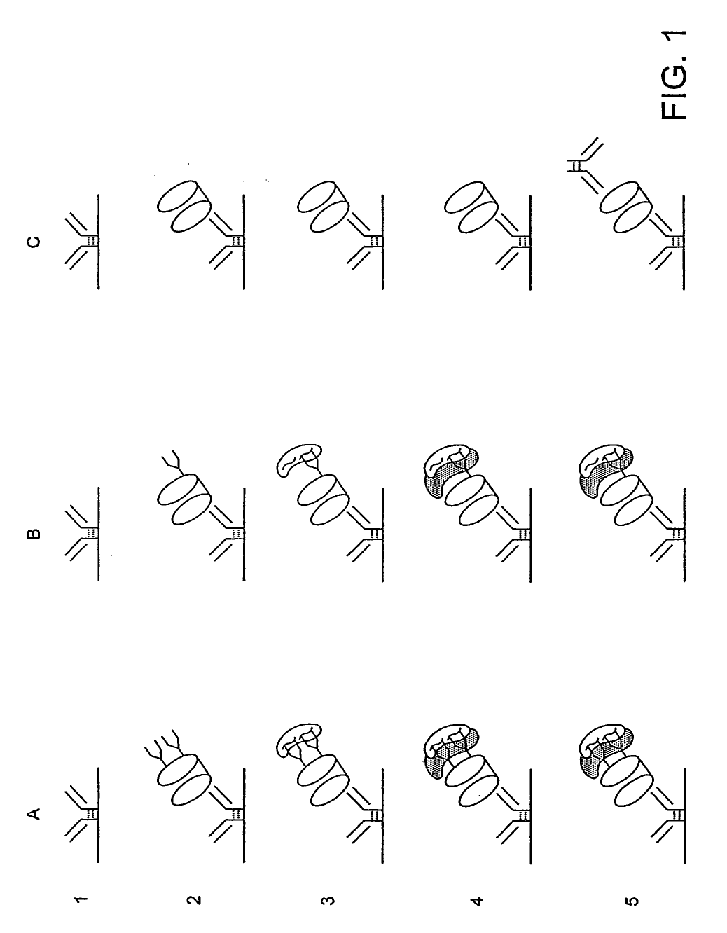

Examples of assays according to the invention for alkaline phosphatase and for transferrin are illustrated schematically in FIGS. 1 to 3 of the accompanying drawings, in which:

FIG. 1 shows schematically the steps (1 to 5) in an assay for asialotransferrin according to the invention;

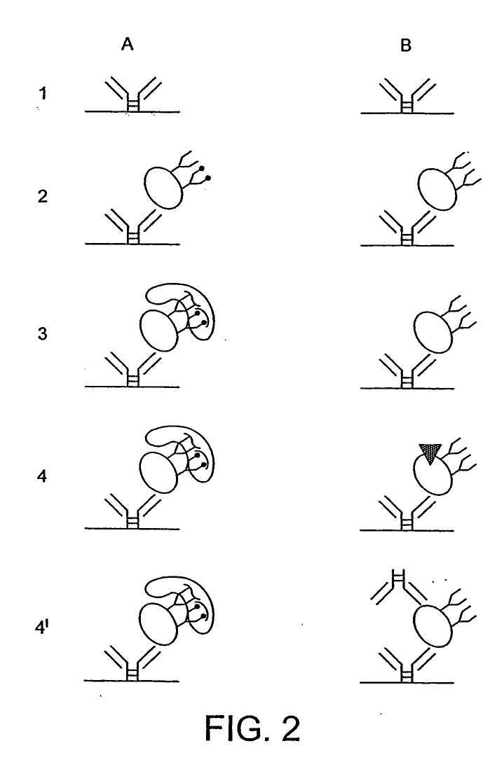

FIG. 2 shows schematically the steps (1 to 4/4′) in an assay for bone and liver alkaline phosphatase according to the invention and

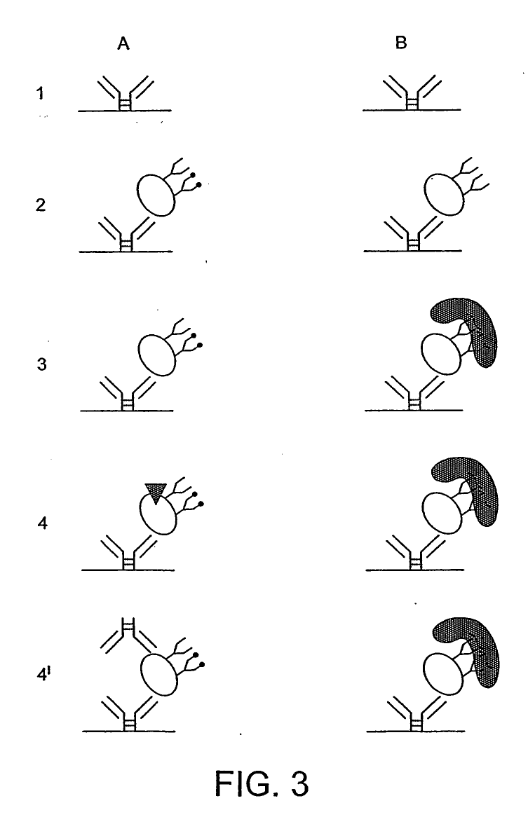

FIG. 3 shows schematically the steps (1 to 4/4′) in an assay for bone and liver alkaline phosphatase according to the invention

In the figures, the columns (three in FIG. 1 and two each in FIGS. 2 and 3) show schematically the interaction of different proteins isoforms with the assay reagents. In FIG. 1, columns A, B and C respectively show the interaction of tetrasialo-disialo- and asialotransferrin. In FIGS. 2 and 3 columns A and B respectively show the interaction of bone and liver alkaline phosphatase.

Referring to FIG. 1, step 1 shows an anti-transferrin antibody immobilized on a surface; in step 2 the transferrin is bound (by capture from the sample); in step 3 a first lectin is added and binds to the carbohydrate in the glycosylated isoforms; in step 4 a further lectin is added; and in step 5 a secondary anti-transferrin antibody to transferrin is added which is only able to bind to the non-lectin bound asialo isoform. The secondary antibody may be labelled to facilitate detection of its complexes.

Referring to FIG. 2, step 1 shows an anti-alkaline phosphatase antibody immobilized on a surface; in step 2 the alkaline phosphatase is bound (by capture from the sample); in step 3 a lectin which can bind to the bone isoform but not the liver isoform is added; and in step 4 a substrate (▴) for alkaline phosphatase is added or alternatively in step 4′ a secondary antibody to alkaline phosphate is added. The enzymatic substrate transformation, or a label on the secondary antibody, allows the amount of liver AP to be measured.

Referring to FIG. 3, step 1 shows anti-alkaline phosphatase antibody immobilized on a surface; in step 2 the alkaline phosphatase is bound (by capture from the sample); in step 3 a lectin which can bind to the liver isoform but not the bone isoform is added; and in step 4 a substrate (▴) for alkaline phosphatase is added or alternatively in step 4 a secondary antibody to alkaline phosphate is added. The enzymatic substrate transformation, or a label on the secondary antibody, allows the amount of bone AP to be measured.

The invention will now be illustrated further with reference to the following non-limiting Examples.

### EXAMPLE 1

Two ligands capable of binding to the N-lobe of transferrin were used. These were bought from Biogenisis, Poole, Dorset, UK and were the full IgG monoclonal antibody referred to as Clone 2A2 and an F(ab)₂ fragment thereof produced by enzyme treatment.

These transferrin binding ligands were coupled to the surface of Biacore chips CMS (Biacore AB, Uppsala, Sweden) using standard amine coupling according to the protocols provided by Biacore. Thus for example the monoclonal antibody (50 μg/mL) was diluted with 0.01M HEPES buffer (pH 7.4 containing 0.15M NaCl, 3 mM EDTA and 0.005% v/v Polysorbate 20) and immobilized on the chip surface using. N-hydroxysuccinimide and N-ethyl-N-dimethylaminopropyl carbodiimide at a 10 μL/min flow rate. All subsequent reagents were injected onto the chip at a 10 μL/min flow rate.

Disialotransferrin and asialotransferrin were isolated from pooled human patients' serum using anion-exchange HPLC. These were then diluted in 0.01M HEPES buffer containing 0.15M NaCl, 3 mM EDTA and 0.005% v/v Polysorbate 20 to concentrations of 58, 5.8, 2.9 and 0.29 μg/mL.

E8 antibody, an antibody specific for the C-lobe of transferrin, (bought from University of Kansas, US (Dr J. D. Cook), and prepared by the method of Guindi et al Am. J. Clin. Nutr. 47:37-41 (1988)), was also diluted in this HEPES buffer to a concentration of 100 μg/mL.

The lectins SNA and ConA (bought from Vector Laboratories, Peterborough, UK and Sigma Aldrich Norway AS, Oslo, Norway respectively), were diluted to concentration of 100 μg/mL in this HEPES buffer to which 5 mM CaCl₂, 5 mM MnCl₂ and 5 mM MgCl₂ had been added. (The presence of divalent cations is often recommended for the stabilization of lectin conformation and binding).

The ligand-coupled Biacore chips were contacted with the transferrin isoform solutions and then sequentially contacted with the SNA and ConA solutions. The relative response units (RU) were then recorded for each transferrin isoform solution—2A2 ligand combination using a Biacore 1000 instrument. The chips were then contacted with the E8 solution and the RU values again recorded.

Enzyme treated F(ab)₂ fragments of Clone 2A2 were also similarly coupled to Biacore chip surfaces.

For the asialotransferrin and disialotransferrin samples, the changes in RU on exposure to E8 were +12.94% and 0.00% respectively which demonstrates the capacity of the assay to distinguish between the differently glycosylated isoforms, and thus to determine the concentration or relative concentration of the differently glycosylated isoforms in admixture.

### EXAMPLE 2

Microtitre Plate Method

The assay is performed in the following stages

1. Add 100 μL of prepared serum samples to capture-antibody coated (e.g. 2A2 coated) Nunc break-apart well strips and incubate with shaking (600/min) for 30 minutes at room temperature.

2. Wash 6 times with 300 μL of 0.05 M phosphate buffered saline (PBS) pH 7.4, containing 0.15 M NaCl, 5 mM MnCl₂, 5 mM CaCl₂ and 0.05% Tween 20 (PBS/Tween20).

3. Add 150 μL of SNA (Sambucus nigra lectin), 0.5 mg/mL in 0.05 M PBS pH 7.4, containing 0.15 M NaCl, 5 mM MnCl₂, 5 mM CaCl₂. Incubate with shaking (600/min) for 20 minutes at room temperature.

4. Aspirate and discard the contents of the wells (do not wash).

5. Add 150 μL of ConA-FITC (Concanavalin ensiformis lectin), 0.5 mg/mL in 0.05 M PBS pH 6.0, containing 0.15 M NaCl, 5 mM MnCl₂, 5 mM CaCl₂. Incubate with shaking (600/min) for 20 minutes at room temperature.

6. Meanwhile, prepare an ¹²⁵I-labelled E8 tracer antibody by diluting as appropriate in 0.05 M PBS pH 7.4, containing 0.15 M NaCl, and 1% BSA (PBS/BSA).

7. Aspirate and discard the contents of the wells (do not wash).

8. Add. 200 μL of ¹²⁵I-labelled tracer antibody diluted as appropriate in PBS/BSA to give around 100000 cpm/200 μL Seal plate to prevent contamination and incubate with shaking (400/min) for 20 minutes at room temperature. In a separate counting vial add 200 μL of ¹²⁵I-labelled E8 for a total cpm count.

9. Remove the seal and invert the plate over absorbent paper and gently tap to remove the fluid. Discard the paper appropriately as radioactive waste.

10. Wash 6 times with PBS/Tween 20, 300 μL per well. Break the wells apart and place into the counting vessels.

### EXAMPLE 3

Microtitre Plate Method

The assay is performed in the following stages

1. Add 100 μL of prepared serum samples to capture-antibody coated (e.g. 2A2 coated) Nunc break-apart well strips and incubate with shaking (600/min) for 30 minutes at room temperature.

2. Wash 6 times with 300 μL of 0.05 M phosphate buffered saline (PBS) pH 7.4, containing 0.15 M NaCl, S mM MnCl₂, 5 mM CaCl₂ and 0.05% Tween 20 (PBS/Tween20).

3. Add 150 μL of SNA (Sambucus nigra lectin), 0.5 mg/mL in 0.05 M PBS pH 7.4, containing 0.15 M NaCl, 5 mM MnCl₂, 5 mM CaCl₂. Incubate with shaking (600/min) for 20 minutes at room temperature.

4. Aspirate and discard the contents of the wells (do not wash).

5. Add 150 μL of ConA-FITC (Concanavalin ensiformis lectin), 0.5 mg/mL in 0.05 M PBS pH 6.0, containing 0.15 M NaCl, 5 mM MnCl₂, 5 M CaCl₂. Incubate with shaking (600/min) for 20 minutes at room temperature.

6. Prepare a 0.1 mg/mL solution of EDC (N-ethyl-N′-(3-dimethyl-amino-propyl)-carbodiimide hydrochloride) in 0.1M PBS, pH7.2 and add 100 μL of this solution to the wells containing the lectins. Incubate with gentle mixing for 20 minutes at ambient temperature.

7. Meanwhile, prepare an ¹²⁵I-labelled E8 tracer antibody by diluting as appropriate in 0.05 M PBS pH 7.4, containing 0.15 M NaCl, and 1% BSA (PBS/BSA).

8. Wash 3 times with PBS/Tween20, 400 μL per well.

9. Add 200 μL of ¹²⁵I-labelled tracer antibody diluted as appropriate in PBS/BSA to give around 100000 cpm/200 μL. Seal plate to prevent contamination and incubate with shaking (400/min) for 20 minutes at room temperature. In a separate counting vial add 200 μL of ¹²⁵I-labelled E8 for a total cpm count.

10. Remove the seal and invert the plate over absorbent paper and gently tap to remove the fluid. Discard the paper appropriately as radioactive waste.

11. Wash 6 times with PBS/Tween 20, 300 μL per well. Break the wells apart and place into the counting vessels.

1. A method for assaying for a protein having at least two isoforms having different glycosylation patterns, said method comprising:
(a) contacting a sample containing said protein with a carbohydrate-binding agent and a primary ligand capable of binding to at least two said isoforms.
(b) subsequent to step (a) contacting said sample with a secondary ligand capable of binding to said protein but not to conjugates of said protein and said carbohydrate-binding agent, and
(c) detecting conjugates of said carbohydrate-binding agent and said protein and/or of said primary ligand and said protein.

2. A method as claimed in claim 1 wherein said carbohydrate-binding agent is a lectin.

3. A method as claimed in claim 1 wherein said protein is selected from transferrin, alkaline phosphatase, chorionic gonadotropin and alpha-fetoprotein.

4. A method as claimed in claim 1 wherein said sample is contacted with said carbohydrate-binding agent and subsequently with said primary ligand.

5. A method as claimed in claim 1 wherein said sample is contacted with said primary ligand and subsequently with said carbohydrate-binding agent.

6. A method as claimed in claim 5 wherein said primary ligand is immobilized on a substrate.

7. A method as claimed in claim 1 wherein said protein is enzymatically active and wherein subsequent to contact with said primary ligand and said carbohydrate-binding agent said sample is contacted with a substrate for the enzymatic action of said protein.

8. A kit for an assay method according to claim 1, said kit comprising a carbohydrate-binding agent, a primary protein binding ligand and a secondary protein binding ligand.

9. A kit as claimed in claim 8 wherein of said ligands at least said primary protein binding ligand is immobilized on a substrate.

10. A kit as claimed in claim 8 further comprising a substrate for the enzymatic activity of the protein to be assayed for using said kit.
